SEQUENCE LISTING (19 sequences)

SEQ ID NO: 1 - DNA, 10 bp, artificial sequence
  designed RBS upstream of target gene
ctaaggagac

SEQ ID NO: 2 - DNA, 29 bp, artificial sequence
  designed ribosomal binding site upstream target gene, e.g. ectA
agacttattctaatctaaggagactaccc

SEQ ID NO: 3 - DNA, 4 bp, Halomonas elongata
  -35 G element of osmotically regulated sigma 38 promoter
gcgg

SEQ ID NO: 4 - DNA, 4 bp, artificial sequence
  artificial, exchanging -35 G element of osmotically regulated sigma 38 promoter
aaat

SEQ ID NO: 5 - DNA, 7 bp, artificial sequence
  -10 element of designed stationary sigma 38 promoter
ctacact

SEQ ID NO: 6 - DNA, 25 bp, artificial sequence
  synthetic spacer of designed stationary sigma 38 promoter
tttctgccaaattccatgaaatcgt

SEQ ID NO: 7 - DNA, 6 bp, Halomonas
  sequence fragment preceding a ribosome binding site
tatgat

SEQ ID NO: 8 - DNA, 8 bp, Halomonas elongata
  sequence fragment preceding an osmotically regulated sigma 38 promoter
ctacatac

SEQ ID NO: 9 - DNA, 8 bp, Halomonas elongata
  sequence fragment following osmo sigma 38 promoter
tttctatt

SEQ ID NO: 10 - DNA, 6 bp, artificial sequence
  consensus sequence of ribosome binding site
aggagg

SEQ ID NO: 11 - DNA, 32 bp, Halomonas elongata
  natural ribosome binding site Halomonas elongata gene HELO_2588
gccgatcaaattcgctacagcgaaccacgaca

SEQ ID NO: 12 - DNA, 24 bp, Halomonas elongata
  osmotically regulated sigma 38 promoter in Halomonas elongata
gcggcctggggagtgggctataat

SEQ ID NO: 13 - DNA, 7 bp, Halomonas elongata
  wt -10 G element of osmotically regulated sigma 38 promoter H. elongata
ctataat

SEQ ID NO: 14 - DNA, 13 bp, Halomonas elongata
  spacer sequence in osmotically regulated sigma 38 promoter of H. elongata
cctggggagtggg

SEQ ID NO: 15 - DNA, 41 bp, Halomonas elongata
  ribosome binding site upstream ectA of H. elongata, with adjacent nucleotides
tatgatgccgatcaaattcgctacagcgaaccacgacaatg

SEQ ID NO: 16 - DNA, 38 bp, artificial sequence
  sequence comprising designed ribosomal binding site with possible adjacent nucleotides
tatgatagacttattctaatctaaggagactacccatg

SEQ ID NO: 17 - DNA, 40 bp, Halomonas elongata
  osmotically regulated sigma 38 with adjacent nucleotides
ctacatacgcggcctggggagtgggctataattttctatt

SEQ ID NO: 18 - DNA, 48 bp, artificial sequence
  sequence comprising designed stationary sigma 38 promoter with possible adjacent nucleotides
ctacatactttctgccaaattccatgaaatcgtctacacttttctatt

SEQ ID NO: 19 - DNA, 127 bp, Halomonas elongata
  transcription initiation sites and putative promoters of the ectoine synthesis gene ectA
ctacatacgaggcctggggagtgggctataatttctattatggaattcagcaagataacctggtttttgaaatgaccataagcggctgttatgatgccgatcaaattcgctacagcgaaccacgaca

(19) United States
(12) Patent Application Publication
(10) Pub. No.: US 2021/0147889 A1
(43) Pub. Date: May 20, 2021

(54) METHOD FOR ENHANCING CONTINUOUS PRODUCTION OF A NATURAL COMPOUND DURING EXPONENTIAL GROWTH PHASE AND STATIONARY PHASE OF A MICROORGANISM

(71) Applicants: bitop AG, Witten (DE); BUNDESREPUBLIK DEUTSCHLAND, VERTRETEN DURCH DIE BUNDESMINISTERIN FUR WIRTSCHAFT UND ENERGIE, DIESE V, Berlin (DE)

(72) Inventors: Hans Jörg Kunte, Kleinmachnow (DE); Gabriela Alfaro Espinoza, Berlin (DE); Andreas Bilstein, Witten (DE)

(73) Assignees: Bundesrepublik Deutschland, Vertreten Durch Die Bundesministerin Für Wirtschaft Und Energie, Berlin (DE); bitop AG, Witten (DE)

(21) Appl. No.: 16/977,129

(22) PCT Filed: Mar. 1, 2018
(86) PCT No.: PCT/EP2018/055077

§ 371 Date: Sep. 1, 2020

(51) Int. Cl. C12P 17/12; C12N 15/67; C12P 7/42

(52) U.S. Cl. CPC C12P 17/12; C12P 7/42; C12N 15/67

(57) ABSTRACT

A method for increased and permanent production of a natural compound by a microorganism during an exponential growth and during a stationary phase of a culture of the microorganism, the method comprising:
enhancing a translation of a target mRNA, wherein the target mRNA comprises a transcript of a target gene, wherein the target gene is encoding the natural compound or a key enzyme involved in a biosynthesis of the natural compound in the microorganism, by replacing a wild type (wt) ribosome binding site (RBS) upstream of the target gene with a synthetic RBS, the synthetic RBS possessing a higher affinity towards a 16S rRNA of a ribosome of the microorganism than the wt RBS; and
enhancing a transcription of the target gene by converting an osmotically regulated σ³⁸ promoter upstream of the target gene into a stationary σ³⁸ promoter, wherein the osmotically regulated σ³⁸ promoter comprises a −35 G-element and a −10 element which are separated from each other by a spacer sequence, by deleting the −35 G element.

### BACKGROUND OF THE INVENTION

Material in an ASCII text file is incorporated herein by reference. The text file is named 20-198_sequence_listing_12-17-2020, the file was created on Dec. 17, 2020, and the file contains 8634 bytes.

The present invention relates to microbiological processes for producing a natural compound. In particular, the invention relates to methods of enhancing the concentration of a biosynthesized substance in the culture medium or in the cytoplasm of cultivated archaea and bacteria during the stationary and exponential growth phase.

As used therein, the term “natural compound” refers to substances such as amino and carboxylic acids and their derivatives—e.g. lysine, proline, trimethylglycine (i.e. glycine-betaine), N,N-dimethyl-L-proline (i.e. proline betaine), diamino butyric acid, Nα-acetyl-L-2,4-diaminobutyrate, ectoine (i.e. 1,4,5,6-tetrahydro-2-methyl-4-pyrimidine carboxylic acid), hydroxyectoine; sugars and their derivatives—e.g. trehalose, glyceryl glucoside; polyols and their derivatives—e.g. erythritol, xylitol; to name a few.

Some of these substances are also known as so called compatible solutes. Compatible solutes are typically synthesized by extremophilic organisms as protectants to mitigate the detrimental effects caused by the extreme environment, e.g. low or high temperature, high salt concentration, low or high pH value. Extremophilic organisms are, e.g., halophilic, thermophilic, or xerophilic archaea, and bacteria. These can thus be used to obtain compatible solutes. As not being disruptive of enzymatic structure or function, even when accumulated in large quantities, compatible solutes are attractive, e.g., for stabilization of bio-functional proteins. For their water-structuring properties and as cell protectants they are used in cosmetics and health care applications. Accordingly, producer strains and appropriately modified cell lines can be used for large-scale production of compatible solutes in fermenters and bio-reactors. The optimization of producer strains and cell lines is the subject of constant effort.

### SUMMARY OF THE INVENTION

In view of the above, according to one aspect a method is suggested for increasing the production of a natural compound by a microorganism during an exponential growth and during a stationary phase of a culture of the microorganism, the method comprising:

enhancing a translation of a target mRNA, wherein the target mRNA comprises a transcript of a target gene, wherein the target gene is encoding the natural compound or a key enzyme involved in a biosynthesis of the natural compound in the microorganism, by replacing a wild type (wt) ribosome binding site (RBS) upstream of the target gene with a synthetic RBS, the synthetic RBS possessing a higher affinity towards a 16S rRNA of a ribosome of the microorganism than the wt RBS; and

enhancing a transcription of the target gene by converting an osmotically regulated σ³⁸ promoter upstream of the target gene into a stationary σ³⁸ promoter, wherein the osmotically regulated σ³⁸ promoter comprises a −35 G-element and a −10 element which are separated from each other by a spacer sequence, by deleting the −35 G element.

According to another aspect, a nucleic acid construct is suggested, comprising: a synthetic ribosome binding site (RBS), a stationary σ³⁸ promoter, a σ⁷⁰ promoter, and a target gene;

the synthetic RBS possessing a higher affinity towards a 16S rRNA of a bacterial ribosome than a wild type (wt) RBS upstream of the target gene;

the stationary σ³⁸ promoter being generated from an osmotically regulated wt σ³⁸ promoter, the osmotically regulated wt σ³⁸ promoter comprising a −35 G-element, by deleting the −35 G-element and/or by replacing the −35 G-element by a nucleotide, the nucleotide being selected from nucleotides others than guanine;

wherein the target gene is encoding a key enzyme, the key enzyme catalyzing a reaction selected from: a phosphorylation/dephosphorylation, a carboxylation/decarboxylation, and an acetylation; the key enzyme being involved in a biosynthesis of a natural compound,

wherein the natural compound is selected from a protein, an amino acid, an derivative of an amino acid, a sugar, and a sugar-polyol.

According to a further aspect, a microorganism is suggested the microorganism being selected from Bacteria and Archaea, wherein a genome of the microorganism encompasses a nucleotide construct, wherein the nucleotide construct comprises: a synthetic ribosome binding site (RBS), a stationary σ³⁸ promoter, a σ⁷⁰ promoter, and a target gene;

the synthetic RBS possessing a higher affinity towards a 16S rRNA of a bacterial ribosome than a wild type (wt) RBS upstream of the target gene;

the stationary σ³⁸ promoter being generated from an osmotically regulated wt σ³⁸ promoter, the osmotically regulated wt σ³⁸ promoter comprising a −35 G-element, by deleting the −35 G-element and/or by replacing the −35 G-element by a nucleotide, the nucleotide being selected from nucleotides others than guanine;

wherein the target gene is encoding a key enzyme, the key enzyme catalyzing a reaction selected from: a phosphorylation/dephosphorylation, a carboxylation/decarboxylation, and an acetylation; the key enzyme being involved in a biosynthesis of a natural compound, wherein the natural compound is selected from a protein, an amino acid, an derivative of an amino acid, a sugar, and a sugar-polyol.

According to yet another aspect, a biotechnical process for continuous production of a natural compound by a microorganism is suggested, wherein a culture of the microorganism is maintained in an exponential growth phase or in a stationary phase, wherein a genome of the microorganism encompasses the nucleotide construct as described above, wherein the microorganism is adapted to excrete the natural compound into a culture medium, the process comprising extracting the natural compound from the culture medium.

### BRIEF DESCRIPTION OF THE DRAWINGS

FIG. 1 shows schematically a comparison of correspondingly aligned ectA RBS and synthetic RBS sequences, together with adjacent nucleic acid sequences. The actual ribosome binding sites are starting each after TATGAT (SEQ ID NO 7) and are ending immediately before the target gene. The target gene in Halomonas elongata starts with ATG.

FIG. 2 shows a sequence comparison of correspondingly aligned wild type osmo σ³⁸ promoters and the new σ³⁸ promoter as suggested herein, both with adjacent nucleic acid sequences. The actual promoters are starting each immediately after CTACATAC (SEQ ID NO 8), and ending before TTTCTATT (SEQ ID NO 9).

FIG. 3 shows the nucleotide sequence comprising two promoters and the RBS upstream of ectA, a sequence comparison of wt σ^{38 osmo} promoter with new and detailed view of fusion products comprising lacZ instead of ectA.

FIG. 4 shows a comparison of β-galactosidase activity obtained with the different nucleotide constructs shown in FIGS. 3C, D, and E, expressed in Miller units, according to Sambrook & Russell, 2001.

FIG. 5 illustrates the ectoine production by strains H. elongata bEH30 and H. elongata KB2.13 during 10 h cultivation.

### DETAILED DESCRIPTION OF THE INVENTION

The continuous production of a given compound is typically preferred in any technology. However, the amount of a compound produced by a given microorganism is highly regulated in response to external factors, e.g. temperature, concentration of certain inorganic ions, ratio of certain inorganic ions, ionic strength, pH-value, nutrients—to name a few. Predominant regulation mechanisms may even differ for different growth phases of the microorganism. Therefore, from an engineering point of view, maintaining an artificial cell culture, e.g. in a bioreactor, in the stationary growth phase, i.e. continuous culture is preferred.

Typically, the synthesis of a natural compound like, e.g., a compatible solute, by a single cell (i.e. biosynthesis) is regulated at different levels. Even though synthesis and catabolism are finally dependent on the cell's genome comprising a DNA sequence, the actual abundance of a certain compound, whether within the cytoplasm or in the external medium, finally depends on the activity of different enzymes. Their abundance and activity, again, depend on the presence and activity of other enzymes, and so forth.

From an engineering point of view, the transcription, i.e. the copying of DNA into mRNA is the first level, where regulation is effectuated. While, for instance, one single operon, i.e. a genomic DNA sequence, controlled by one (or several) promoter(s), suffices for a certain compound (e.g. in a certain strain), for another compound (or another strain) a more complex organization of several gene clusters under the control of different promoters may be possible. Moreover, the initiation of the synthesis of an enzyme initially depends on the binding of RNA polymerase to the DNA strand, i.e. on the very first stage of the so-called transcription. As used herein, a promoter is a regulatory region of DNA located upstream of a gene, the gene encoding the key compound, providing a control point for regulated gene transcription.

Transcription proceeds in the following general steps (cf https://en.wikipedia.org/wiki/Transcription_(biology)):
1. RNA polymerase, together with one or more general transcription factors, binds to promoter DNA.
2. RNA polymerase creates a transcription bubble, which separates the two strands of the DNA helix. This is done by breaking the hydrogen bonds between complementary DNA nucleotides.
3. RNA polymerase adds RNA nucleotides (which are complementary to the nucleotides of one DNA strand).
4. RNA sugar-phosphate backbone forms with assistance from RNA polymerase to form an RNA strand,
5. Hydrogen bonds of the RNA-DNA helix break, freeing the newly synthesized RNA strand.
6. If the cell has a nucleus, the RNA may be further processed. This may include polyadenylation, capping, and splicing.
7. The RNA may remain in the nucleus or exit to the cytoplasm through the nuclear pore complex.

The strand of DNA transcribed into an RNA molecule is called a transcription unit and encodes at least one gene. If the gene encodes a protein, the transcription produces a so-called messenger RNA (mRNA). The mRNA, in turn, serves as a template for the synthesis of the protein (enzyme) through translation.

On the other hand, the mentioned translation, i.e. the process in which ribosomes in the cytoplasm synthesize proteins after the transcription of DNA to RNA (cf. https://en.wikipedia.org/wiki/Translation_(biology)) also regulates the expression of the compound. The entire process is called gene expression. In translation, the mRNA is decoded by a ribosome to produce a specific amino acid chain, or polypeptide. The ribosome facilitates decoding by inducing the binding of complementary tRNA anticodon sequences to mRNA codons. The tRNAs carry specific amino acids that are chained together into a polypeptide as the mRNA passes through the ribosome and is consecutively “read” by the ribosome.

The translation proceeds in three phases:
1. Initiation: The ribosome assembles around the target mRNA. The first tRNA is attached at the start codon.
2. Elongation: The tRNA transfers an amino acid to the tRNA corresponding to the next codon. The ribosome then moves (translocates) to the next mRNA codon to continue the process, creating an amino acid chain.
3. Termination: When a stop codon is reached, the ribosome releases the polypeptide.

Therein, the initiation generally starts at a ribosome binding site, or ribosomal binding site (RBS), which is a sequence of nucleotides upstream of the start codon of an mRNA transcript that is responsible for the recruitment of a ribosome. Typically, a RBS in the DNA of Bacteria comprises a purine rich, so called Shine-Dalgarno (SD) sequence. It is generally acknowledged that base pairing between the SD sequence in mRNA and the 3′ end of 16S rRNA is of prime importance for initiation of translation by bacterial ribosomes.

As evident, the abundance of any natural compound which is produced in a multi-step biosynthesis by enzymatically transforming e.g. an amino acid precursor by an enzyme cascade into an amino acid derivative is regulated by an even more complex cascade of regulatory mechanisms, since the abundance of each of the transforming enzymes is regulated at the level of transcription and translation as well.

Therefore, the manipulation of the metabolism of a microorganism to generate more efficient producer strains is quite challenging. It is further complicated since, e.g., published gene sequences of respective microorganisms may be incorrect. Furthermore, the assignment of putative initiation codons may be incorrect.

In view of the above, different approaches have been followed to establish enhanced production of compatible solutes, e.g. of ectoine, which is synthesized and accumulated as an osmolyte by several halophilic microorganisms.

In particular, halophilic microorganisms from genera like Bacillus, Brevibacterium, Chromohalobacter, Halobacillus, Halomonas, Marinococcus, Methylomicrobium and transgenic or recombinant microorganisms, e.g. Corynebacterium glutamicum, Escherichia coli, Bacillus subtilis, may be used for the industrial production of compatible solutes, like, e.g. ectoine.

The following concepts have been developed and employed to increase ectoine yield. First of all, disruption of ectoine transport, which leads to over-production and excretion of ectoine to the medium, secondly, mutation of the degradation pathway of ectoine, which further increases ectoine synthesis, and finally, feeding with toxic solute analogues, which facilitates to select for ectoine overproduction and excretion strains.

In contrast to previous concepts, we herein suggest a new approach by combining both transcriptional and translational up-regulation.

To exemplarily study the possibility of improving the production of compatible solutes we choose the halophilic bacterium Halomonas elongata. It amasses compatible solutes in the cytoplasm to achieve an osmotic equilibrium with the environment (Kunte, 2006; Schwibbert et al., 2011). Particularly, the compatible solute synthesized by H. elongata is ectoine, an amino acid derivative of aspartate. This bacterium utilizes three enzymes (EctA, EctB, and EctC) for de novo synthesis of ectoine (Schwibbert et al., 2011). The genes encoding these three enzymes are ectABC, which are transcriptionally regulated by three different promoters.

Upstream of ectA there is a sigma factor σ⁷⁰ promoter and an osmotically induced σ^{38-osmo} promoter. Upstream of ectC there is a σ⁵⁴ promoter. σ⁵⁴-controlled promoters are often involved in transcription of nitrogen-regulated genes. These findings indicate that ectoine synthesis is regulated by salinity (σ^{38-osmo}), growth (σ⁷⁰), and nitrogen supply (σ⁵⁴) as described by Schwibbert et al., (2011).

Ectoine is used by the H. elongata cell not only as osmolyte but—depending on life conditions—also as an energy source. In the corresponding catabolism four further enzymes DoeA, DoeB, DoeC and DoeD are involved (cf. Schwibbert et al., 2011).

According to the approach suggested here, firstly the key enzyme involved in the biosynthesis of ectoine has been identified for favorably shifting the equilibrium in the complex metabolic pathway of ectoine towards its enhanced synthesis. It is believed that L-2,4-diaminobutyric acid Nγ-acetyltransferase is the key enzyme in ectoine biosynthesis. This enzyme is encoded by the gene ectA. In the following text, the gene which encodes the key enzyme will generally be called target gene. Enzymes that make a certain metabolic pathway irreversible are considered key enzymes. These enzymes catalyze reactions such as phosphorylation/dephosphorylation, carboxylation/decarboxylation and acetylation.

Thus, according one embodiment, in order to enhance the abundance and thereby the production of such a natural compound like, e.g., ectoine, by the cultivated cells we suggest applying a combinatorial approach of optimizing expression at the level of transcription (mRNA synthesis) and translation (protein synthesis) simultaneously.

According to exemplary embodiments, in order to improve the expression of the natural compound, e.g. ectoine, the RBS upstream of the target gene, encoding the key enzyme, e.g. ectA, can be determined by analyzing the genome of a promising producer microorganism, e.g. of H. elongata. The RBS in front of ectA differs strongly from the consensus sequence known from literature (AGGAGG; SEQ ID NO 10). In particular, the wt RBS of H. elongata appears a weak binding site for ribosomes, as it substantially deviates from the SD sequence, known for bacteria. Usually, the RBS is rich in guanine G and adenine A and the SD motif is typically AGGAGG. The wt sequence strongly deviates from known motifs, indicating that ectA-mRNA is only weakly translated into the corresponding key enzyme (L-2,4-diaminobutyric acid Nγ-acetyltransferase), which is encoded by the gene downstream of the RBS.

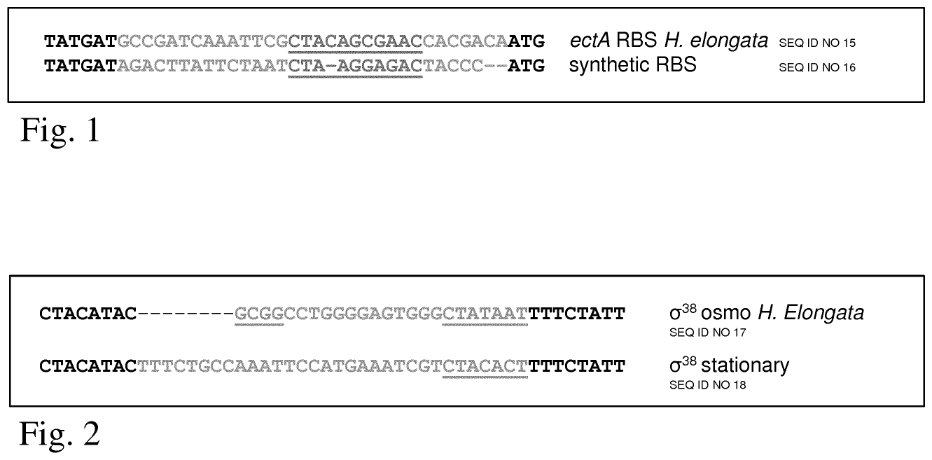

Thus, in order to improve translation, we developed a synthetic RBS (FIG. 1) using the library calculator software (Espah Borujeni et al., 2014; Salis et al., 2009; https://salislab.net/software/RBSLibraryCalculatorEvaluateMode). Typically, using the library calculator software comprises inputting into the calculator software a nucleotide sequence upstream of the wt RBS and inputting at least a sequence of nucleotides of the target gene. The library calculator software delivers a sequence of a synthetic RBS which possesses a higher affinity towards the 16S rRNA than the wt RBS. This new, i.e. synthetic RBS is specifically designed for optimal translation of the ectA mRNA and hence, results in up-regulating the translation of the corresponding gene, in comparison to the corresponding strain with the natural (wt) RBS.

In addition thereto, enhancing transcription of the target mRNA, which mRNA is a transcript of the target gene, is reached by modifying a natural promoter upstream of the target gene. The target gene encodes the key enzyme which is involved in the biosynthesis of the natural compound.

In particular, according to this embodiment, the σ^{38 osmo} promoter of H. elongata is modified by deleting its −35 G-element. In particular, the nucleotide sequence GCGG (SEQ ID NO 3) is deleted. The −35 G-element is the characteristic motif that distinguishes σ^{38 osmo} promoters from σ³⁸ promoters. σ^{38 osmo} promoters can be changed into σ³⁸ by either deleting the G-element or by replacing it by any non-G containing DNA. The −10 region can remain unchanged as both sigma promoters share the same consensus sequence. If the −10 region deviates strongly from the sigma consensus sequence (CTACACT; SEQ ID NO 5), the strength of sigma promoters can be increased however by changing the −10 region and making it more similar to the −10 consensus motif. (Lee & Gralla, 2001).

According to the suggested approach, we do not just introduce an artificial or viral promoter for strong transcription as promoters of this type often have negative effects on cell health, product quality (e.g. formation of inclusion bodies) and volumetric productivity.

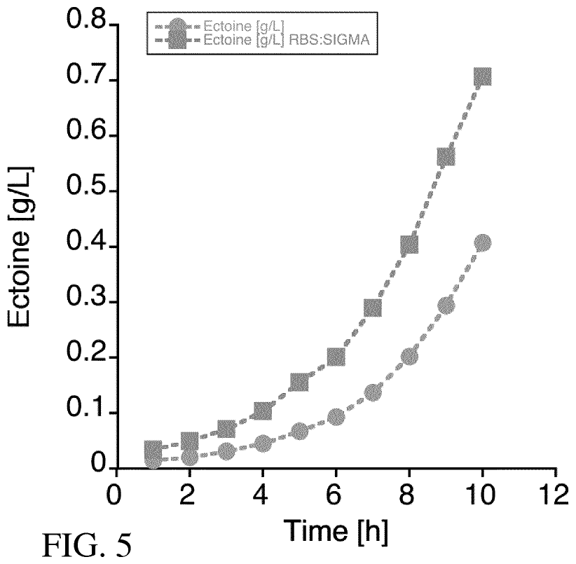

Contrary thereto, as mentioned above, we suggest modifying a wild type (wt) promoter. The wild type σ^{38-osmo} promoter comprises −10 and −35 sequences and in that respect, it resembles σ⁷⁰ dependent promoters (cf. FIG. 5 and Schwibbert et al., 2011). The −10 and −35 sequences are separated by 17 bp, a typical spacing for promoters controlled by σ⁷⁰.

Thus, according to an embodiment, we suggest removing the −35 G-element of wt osmo σ³⁸. Surprisingly, deleting the −35 G element causes extending transcription from growth phase into stationary phase and at the same time allows for optimal translation.

Thus, our approach is exemplified by the changes introduced at the ectA site. The σ⁷⁰ promoter allows for transcription during growth phase and remains untouched. The σ^{38-osmo} promoter is induced by potassium (salt stress). In order to extend transcription, the −35 region (G-box) of the osmotically induced promoter is either removed without any substitution or replaced by nucleotides other than G. This changes the promoter into a stationary phase σ³⁸ promoter. These changes are physiological compatible with the cell and have no effect on growth and increase transcription (cf. FIG. 5).

Favorably, the suggested modifications are:
i) physiologically compatible and
ii) encompassed with merely minimal changes to the genetic makeup of the cell (no GMO).

Such combinatorial approach of enhancing the production of a natural compound by a microorganism, comprising up-regulating both at the level of transcription and translation, has not been even envisioned before.

Applying our approach can be used to improve
i) protein expression,
ii) synthesis of amino acids and derivatives, and
iii) synthesis of compatible solutes such as sugars, sugar-polyols, amino acids and amino acid derivatives.

The methodology is feasible in any bacteria but preferable in extremophilic bacteria where genes are under the dual control of a vegetative σ⁷⁰ promoter and a stress promoter (e.g. heat or osmo stress promoters) and where the RBS is weak. Favorably, improving transcription and translation as described will only introduce minor changes to the genome of the target organism. Since the changes are minor, the resulting organisms are not regarded as genetically modified (no GMO) and can be used for a wide field of application in industry.

As experimentally demonstrated, combining subtle promoter modifications, which closely resemble the natural occurring promoters in the target organism, with a modification of the RBS lead to a dramatic increase in expression of ectA and subsequently of the entire ectoine production (cf. FIG. 5).

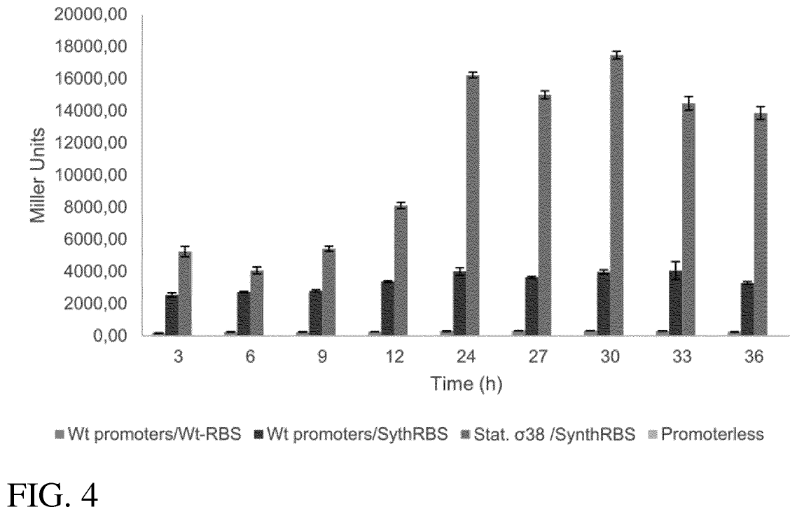

Surprisingly, optimizing the RBS alone (leaving the wt promoters unchanged) already improved expression. This has been observed experimentally by a β-Galactosidase assay, based on the expression of the gene lacZ, which encodes the enzyme β-galactosidase in Halomonas elongata. This enzyme cleaves lactose, a disaccharide, into glucose and galactose. The enzyme activity can easily be quantified (cf. FIG. 4). After 24 h of cultivation a mean density of cultivated cells is reached which corresponds to a stationary phase of the batch culture. The mean density of cells can be measured by detecting an optical density (OD₆₀₀) of the cell suspension at 600 nm (not shown).

### A. First Example

In order to make the ectoine synthesis process more efficient, the H. elongata strain KB2.13 was used. This strain synthesizes more ectoine than the wild-type strain and excretes ectoine into the medium (ΔteaABC, ΔdoeA). Strain KB2.13 not only produces more ectoine than H. elongata DSM 2581^{T} but also reduces expenses during industrial processes (Grammann et al., 2002; Kunte et al., 2014).

In order improve the translation in comparison to the wild type, a synthetic ribosomal binding site (synthetic RBS) upstream of the target gene ectA was designed using the library calculator software (Espah Borujeni et al., 2014; Salis et al., 2009). In case of the used H. elongata, the target gene ectA comprises the start codon ATG.

The natural RBS located upstream of ectA comprises the sequence (SEQ ID NO 11):

GCCGATCAAATTCGCTACAGCGAACCACGACA

(cf. FIG. 1). The actual binding site is underlined and written in bold. The sequence between the binding site and the beginning of the gene is written in cursive and is called spacer. The length of the spacer influences the efficiency of the RBS and translation.

The designed nucleotide sequence of the synthetic RBS comprises the sequence (SEQ ID NO 2):

AGACTTATTCTAATCTAAGGAGACTACCC

(cf. FIG. 1). The actual binding site is underlined and written in bold. The sequence between the binding site and the beginning of the target gene is written in cursive and is called spacer. The length of the spacer influences the efficiency of the RBS and translation.

The synthetic RBS sequence was transcriptionally fused with the reporter gene lacZ encoding the enzyme β-Galactosidase, cloned in the vector pBBR1MCS (Kovach et al., 1995), and transferred to the H. elongata cells via E. coli ST18 or E. coli 517-1 mediated conjugation (Grammann et al. 2002; Schwibbert et al. 2011). Bacterial conjugation is the transfer of genetic material such as plasmids by cell to cell contact via a bridge like connection (here: E. coli to H. elongata). Conjugation has the advantage over other transfer methods that it is very efficient in transferring DNA and allows transfer of large amounts of DNA. For further quantification, the same procedure described in the second embodiment was used (see below).

### B. Second Example

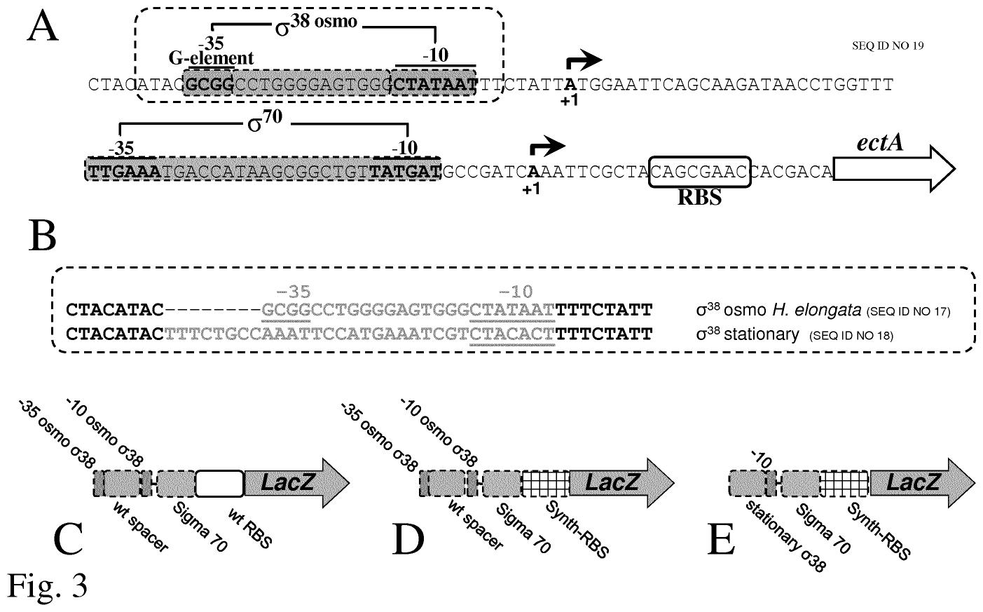

In order to further enhance the ectoine production, even at the level of transcription, the osmotically regulated σ³⁸ promoter of the wild type was edited by removing the −35 G-element (cf. FIGS. 2 and 3) and exchanged for a stationary σ³⁸ promoter, designated also as σ^{38-stationary} (cf. FIG. 2).

In particular, the new σ³⁸ promoter as shown in FIG. 2 has been experimentally proven to activate transcription during the stationary growth phase of H. elongata. Therefore, we designate it as σ³⁸ stationary. As used herein, a σ³⁸stationary promoter is a promoter which is the main promoter during the stationary growth phase of the microorganism, i.e. bioconversion by slow-growing or non-growing microorganisms. Accordingly, this stationary σ³⁸ promoter allows for continuous transcription of the gene which follows downstream, e.g. ectA, lacZ or another target gene.

The sequence of the σ^{38-osmo} promoter of H. elongata comprises a sequence (SEQ ID NO 12): GCGGCCTGGGGAGTGGGCTATAAT. The G-element GCGG (SEQ ID NO 3) at −35 and the −10 sequence are written in bold. The spacer that separates the −35 and −10 region is written in cursive. As indicated above σ^{38-osmo} regulates the transcription of ectA, and hence the biosynthesis of ectoine during the growth phase under osmotic stress.

In contrats thereto, the sequence of the stationary σ³⁸ comprises CTACACT (SEQ ID NO 5) as shown in FIG. 2 (σ³⁸ stationary) and enhances ectoine production by Halomonas elongata. The G-element at −35 was replaced by AAAT (SEQ ID NO 4).

The synthetic RBS sequence was transcriptionally fused with the reporter gene lacZ encoding the enzyme β-Galactosidase, cloned in the vector pBBR1MCS (Kovach et al., 1995), and transferred to H. elongata via E. coli mediated conjugation. The same was done for the stationary σ³⁸ promoter (FIG. 3) (Lee & Gralla, 2001, 2004) as well as the wild-type RBS and wild-type σ³⁸ promoter from ectA gene. In total three different transcriptional fusions were constructed and tested in comparison to a promoterless control (Table 1, FIG. 3 and FIG. 4).

TABLE 1
RBS, σ³⁸ and σ⁷⁰ promoters transcriptionally
fused with the lacZ gene.

| | |
|---|---|
| 3 (C) | Wild-type- ectA_promoters (σ^{38-osmo} and σ⁷⁰) + |
| | Φ(wild-type RBS-lacZ) |
| 3 (D) | Wild-type- ectA_promoters (σ^{3-osmo8} and σ⁷⁰) + |
| | Φ(synthetic RBS-lacZ) |
| 3 (E) | σ^{38-stationary} promoter + σ⁷⁰ + |
| | Φ(synthetic RBS-lacZ) |

Therein, Φ refers to the fusion sequence of the specified RBS with the lacZ gene. To determine the activity of the new promoter and synthetic RBS, β-Galactosidase assays (Sambrook & Russell, 2001) were performed in MM63 media (Larsen et al 1987), at 30° C., and 155 rpm. A total of four replicates were used per treatment. The assays revealed that the synthetic RBS and stationary promoter σ^{38-stationary} enhances the expression of gene lacZ by a factor of approximately 20 (FIG. 3).

In particular, FIG. 3A shows the arrangement of natural promoters σ^{38 osmo} and σ⁷⁰ as well as the position of the ribosome binding site (RBS) within the nucleotide sequence of Halomonas elongata DSM 2581^{T}. Both the promoters and the RBS regulate transcription and translation of the gene ectA (Helo_2588; https://www.ncbi.nlm.nih.gov/), respectively (modified according to Schwibbert et al., 2011). Transcription initiation sites are marked with an arrow and (+1). The σ⁷⁰ promoter (pink) allows for transcription of the gene following downstream during the exponential growth phase. The promoter σ^{38osmo} activates the transcription under osmotic stress, which is recognized by an enhanced concentration of potassium ions. Nucleotides of the GA-rich RBS are marked by a solid black frame. The sequence deviates from the previously known motif, which typically characterizes an RBS in Bacteria. The observed deviation indicates that the translation of ectA-mRNA into protein (key enzyme) is mitigated.

FIG. 3B shows a comparison of the natural nucleotide sequence of σ^{38osmo} as marked in part A and the stationary promoter σ³⁸ from H. elongata. The promoter σ^{38osmo} and the suggested herein stationary σ³⁸ are identical with respect to their −10 region. However, the stationary σ³⁸ is lacking the G-element at position −35. The deletion of the G-element transforms σ^{38osmo} into a σ³⁸-similar promoter, which has experimentally been proven to activate transcription during stationary growth phase. Since the synthesis of ectoine is almost completely discontinued in the stationary phase in a batch culture of H. elongata, the removal of the G-element promises to intensify ectA expression and thus increase the synthesis of ectoine.

FIGS. 3C, D and E show schematically different lacZ-fusion constructs, where the wt nucleotides of gene ectA have been replaced by lacZ. The gene lacZ was thus expressed (C) under the control of natural promoters (wt σ^{38 osmo}, wt σ⁷⁰) and natural RBS (wild type). In another construct (D) the natural promoter was linked to the synthetic RBS (up-regulation at the level of translation). In yet another construct (E) the developed stationary promoter σ³⁸ and the synthetic RBS were fused for optimization of both transcription and translation.

After determining the activity of the different promoters and RBS, the wild type promoter σ^{38-osmo} and the natural RBS were removed from the chromosome of H. elongata KB2.13 and replaced by the stationary promoter σ^{38-stationary} and the synthetic RBS. The resulting strain was named H. elongata bEH30.

Then, ectoine synthesis was measured and quantified via HPLC in the KB2.13 strain and bEH30 strain. For this, both strains were cultured at 33° C., 250 rpm in 20 mL MOPS-buffered minimal medium. Growth and glucose consumption of both strains were identical. Ectoine is excreted to the medium and was measured by HPLC every hour during a period of 10 h. Introducing a stationary promoter σ^{38-stationary} and a synthetic RBS upstream of ectA in H. elongata increased ectoine production. The newly developed strain bEH30 (FIG. 5 RBS:SIGMA) produced 0.707 g/L ectoine while the regular strain reached only a concentration of 0.4075 g/L ectoine. This corresponds to an increase in volumetric productivity of 70% (cf. FIG. 5).

In particular, FIG. 5 shows the ectoine production by Halomonas elonagta with modified RBS and promoter σ³⁸ (blue) in comparison to a regular production strain of H. elongata (orange). Both strains were grown in 20 mL MOPS-buffered minimal medium for 10 h at 33° C. The product ectoine is excreted to the medium and measured by HPLC. The data shown are the mean of two independent experiments. Growth and glucose consumption of both strains were identical. The newly developed strain, designated in FIG. 5 as “RBS:SIGMA” produced 0.707 g/L ectoine while the regular strain reached only a concentration of 0.4075 g/L ectoine.

As shown above, in order to improve ectoine production, the osmo σ³⁸ promoter) (σ^{38-osmo}) and the natural RBS located upstream of ectA were exchanged for a stationary σ³⁸ promoter (σ^{38-stationary}) and a synthetic RBS, respectively. The methods described allow for enhanced gene expression by a synthetic ribosomal binding site (RBS) in combination with an optimized σ³⁸ promoter for increased synthesis of proteins, amino acids, amino acid derivatives, sugars and sugar-polyols in extremophilic Bacteria and Archaea.

In view of the above, according to an embodiment, a method for permanent production of Nγ-acetyl-L-2,4-diaminobutyrate and/or ectoine by Halomonas elongata during an exponential growth and during a stationary phase of a cultivated strain of Halomonas elongata is suggested. This method comprises the steps of:
identifying L-2,4-diaminobutyric acid Nγ-acetyltransferase to comprise a key enzyme in the biosynthesis of Nγ-acetyl-L-2,4-diaminobutyrate and of ectoine by Halomonas elongata;
identifying a target gene, the target gene encoding the key enzyme;
detecting a wild type ribosome binding site (wt RBS) upstream of the target gene;
designing a synthetic RBS, wherein the synthetic RBS possesses a higher affinity towards a 16S rRNA of Halomonas elongata than the wt RBS;

and
detecting an osmotically regulated σ³⁸ promoter upstream of the target gen, the osmotically regulated σ³⁸ promoter comprising a −35 G-element and a −10 element which are separated from each other by a spacer nucleotide sequence,
modifying the osmotically regulated σ³⁸ promoter by deleting the −35 G element;
generating the strain of Halomonas elongata by replacing in the genome of Halomonas elongata the wt RBS by the synthetic RBS, and by replacing the osmotically regulated σ³⁸ promoter by the modified σ³⁸ promoter; and
cultivating Halomonas elongata comprising the strain and maintaining stationary growth, wherein maintaining stationary growth encompasses keeping a mean cell density of Halomonas elongata in a batch culture constant.

Herein, the first four steps effectuate an enhanced translation of the genetic information related to the biosynthesis of ectoine and at least of Nγ-acetyl-L-2,4-diaminobutyrate, and comprise an up-regulation of their biosynthesis at the level of translation. The next four steps effectuate an enhanced transcription and hence, comprise up-regulating the biosynthesis of the mentioned compounds at the level of transcription.

As can be easily contemplated, the suggested approach is not only applicable to halophilic microorganisms, like, e.g., Halomonas elongata, but can also be adapted to other halophilic Bacteria and Archaea, or other biotechnically cultivated cells, wherein the cultivated cells are modified to have available genetic information concerning the production of a natural compound of interest. In particular, the genome of the cultivated cell contains at least a nucleotide sequence comprising the described above stationary σ³⁸ promoter (σ^{38-stationary}) and a synthetic RBS upstream of a target gene, which encodes either the natural compound of interest or a key enzyme, wherein the key enzyme is involved in the biosynthesis of the natural compound of interest. Advantageously, the genome of the cultivated cell further comprises, between the stationary σ³⁸ promoter and the synthetic RBS a σ⁷⁰ promoter.

In view of the above, the target gene (key gene) may also be selected to contain a start codon which is different from ATG, (such as GTG, TTG or CTG), depending on the natural compound of interest.

According to further embodiments, we herewith suggest:
a) A method for permanent production of ectoine by Halomonas elongata during an exponential growth and during a stationary phase of a cultivated strain of Halomonas elongata, the method comprising:
   identifying L-2,4-diaminobutyric acid Nγ-acetyltransferase to comprise a key enzyme in the biosynthesis of ectoine by Halomonas elongata;
   identifying a target gene, the target gene encoding the key enzyme;
   detecting a wild type ribosome binding site (wt RBS) upstream of the target gene;
   designing a synthetic RBS, wherein the synthetic RBS possesses a higher affinity towards a bacterial 16S rRNA than the wt RBS; towards a 16S rRNA of H. elongata;
and
   detecting an osmotically regulated σ³⁸ promoter upstream of the target gene, the osmotically regulated σ³⁸ promoter comprising a −35 G-element and a −10 element which are separated from each other by a spacer,
   modifying the osmotically regulated σ³⁸ promoter by deleting the −35 G element;
   generating the strain of Halomonas elongata by replacing in the genome of Halomonas elongata the wt RBS by the synthetic RBS, and the osmotically regulated σ³⁸ promoter by the modified σ³⁸ promoter; and
   cultivating Halomonas elongata comprising the strain and maintaining stationary growth.
b) The method according to embodiment a), wherein the wt RBS comprises the nucleotide sequence GCCGATCAAATTCGCTACAGCGAACCACGACA (SEQ ID NO 11) and the synthetic RBS comprises the nucleotide sequence AGACTTATTCTAATCTAAGGAGACTACCC (SEQ ID NO 2).
c) The method according to embodiments a) or b), wherein the −35 G element comprises the nucleotide sequence GCGG (SEQ ID NO 3), the −10 element comprises the nucleotide sequence CTATAAT (SEQ ID NO 13), and the spacer comprises the nucleotide sequence CCTGGGGAGTGGG (SEQ ID NO 14).
d) The method according to any of embodiments a) through c), wherein the modified σ³⁸ promoter is a stationary promoter, the genome of Halomonas elongata further comprising a σ⁷⁰ promoter, which allows for transcription of the target gene during a growth phase of the strain of Halomonas elongata.
e) The method according to any of embodiments a) through d), wherein the spacer is replaced by the nucleotide sequence TTTCTGCCAAATTCCATGAAATCGT (SEQ ID NO 6).
f) The method according to any of embodiments a) through e), wherein Halomonas elongata is adapted to release ectoine into a culture medium, the method further comprising:
   extracting the ectoine from the culture medium.
g) A nucleotide construct comprising a target gene; a synthetic RBS; a promoter which allows for transcription of a target gene, the target gene encoding L-2,4-diaminobutyric acid Nγ-acetyltransferase, during an exponential growth phase and a stationary phase of a halophilic microorganism; and a modified σ³⁸ promoter, the modified σ³⁸ promoter being generated by deleting a −35 G-element of an osmotically regulated wt σ³⁸ promoter or by replacing the −35 G-element of the osmotically regulated wt σ³⁸ promoter by a nucleotide, wherein the nucleotide is selected from nucleotides others than guanine.
h) The nucleotide construct according embodiment g), wherein the synthetic RBS, the σ⁷⁰ promoter, and the modified σ³⁸ promoter are arranged in an upstream direction of the target gen.
i) The nucleotide construct according to embodiment g) or h), wherein the target gene is encoding a natural compound selected from: an amino acid, an amino acid derivative, a carboxylic acid, a carboxylic acid derivative, a sugar, a sugar derivative, a polyol, a derivative comprising a polyol; or a key enzyme involved in the biosynthesis of the natural compound.
j) The nucleotide construct according to any of embodiments g) through i), wherein the promoter which allows for transcription of the target gene during the growth phase of the halophilic microorganism is the σ⁷⁰ promoter of Halomonas elongata.

Thus, according to an embodiment (1), a method for increased and permanent production of a natural compound by a microorganism during an exponential growth and during a stationary phase of a culture of the microorganism is suggested. The method comprises: enhancing a translation of a target mRNA, wherein the target mRNA comprises a transcript of a target gene, wherein the target gene is encoding the natural compound or a key enzyme involved in a biosynthesis of the natural compound in the microorganism, by replacing a wild type (wt) ribosome binding site (RBS) upstream of the target gene with a synthetic RBS, the synthetic RBS possessing a higher affinity towards a 16S rRNA of a ribosome of the microorganism than the wt RBS; and enhancing a transcription of the target gene by converting an osmotically regulated σ³⁸ promoter upstream of the target gene into a stationary σ³⁸ promoter, wherein the osmotically regulated σ³⁸ promoter comprises a −35 G-element and a −10 element which are separated from each other by a spacer sequence, by deleting the −35 G element.

According to an embodiment (2), the mentioned −35 G element of the osmotically regulated wt σ³⁸ promoter is deleted by replacing it with nucleotides others than guanine (G).

According to an embodiment (3), the method of embodiment (1) or (2) further comprises: cultivating the microorganism, wherein a genome of the cultivated microorganism encompasses in a downstream direction the stationary σ³⁸ promoter, a σ⁷⁰ promoter, the synthetic RBS, and the target gene.

According to an embodiment (4), in the method according to any of embodiments 1-3 the natural compound is selected from a protein, an amino acid, an derivative of an amino acid, a sugar, and a sugar-polyol.

According to an embodiment (5), in the method according to any of embodiments 1-4 the microorganism is a halophilic microorganism and the natural compound is selected from a compatible solute, the compatible solute being selected from a sugar, a sugar-polyol, an amino acid and an amino acid derivative.

According to an embodiment (6), in the method according to embodiment (4) the natural compound is selected from Nγ-acetyl-L-2,4-diaminobutyrate, ectoine, and hydroxyectoine.

According to an embodiment (7), in the method according to embodiment (6) the microorganism is Halomonas elongata and the key enzyme is L-2,4-diaminobutyric acid Nγ-acetyltransferase.

According to an embodiment (8), in the method according to embodiment (7) the synthetic RBS comprises the nucleotide sequence CTAAGGAGAC (SEQ ID NO 1).

According to an embodiment (9), in the method according to embodiment (8) the synthetic RBS comprises the nucleotide sequence

(SEQ ID NO 2)
AGACTTATTCTAATCTAAGGAGACTACCC.

According to an embodiment (10), in the method according to embodiment (9) the −35 G-element of the osmotically regulated σ³⁸ promoter comprises the nucleotide sequence GCGG (SEQ ID NO 3), wherein converting the stationary regulated σ³⁸ promoter into the stationary σ³⁸ promoter comprises replacing GCGG (SEQ ID NO 3) with AAAT (SEQ ID NO 4).

According to an embodiment (11), in the method according to embodiment (10) the nucleotide sequence of the stationary σ³⁸ promoter comprises the nucleotide sequence

(SEQ ID NO 6)
TTTCTGCCAAATTCCATGAAATCGTCTACACT.

According to an embodiment (12), in the method according to embodiment (11) the nucleotide sequence of the stationary σ³⁸ promoter comprises the nucleotide sequence

(SEQ ID NO 5)
CTACACT.

According to an embodiment (13), a nucleotide construct is suggested, wherein the nucleotide construct comprises: a synthetic ribosome binding site (RBS), a stationary σ³⁸ promoter, a σ⁷⁰ promoter, and a target gene; wherein the synthetic RBS upstream of the target gene possesses a higher affinity towards a 16S rRNA of a bacterial ribosome than a wild type (wt) RBS towards the 16S rRNA of the bacterial ribosome; wherein the stationary σ³⁸ promoter is generated starting from an osmotically regulated wt σ³⁸ promoter, the osmotically regulated wt σ³⁸ promoter comprising a −35 G-element, by deleting the −35 G-element and/or by replacing the −35 G-element by a nucleotide, wherein the nucleotide is selected from nucleotides others than guanine (G); wherein the target gene is encoding a key enzyme, the key enzyme catalyzing a reaction selected from: a phosphorylation/dephosphorylation, a carboxylation/decarboxylation, and an acetylation; the key enzyme being involved in a biosynthesis of a natural compound, wherein the natural compound is selected from a protein, an amino acid, a derivative of an amino acid, a sugar, and a sugar-polyol.

According to an embodiment (14), in the nucleotide construct according to embodiment (13) the −35 G-element of the osmotically regulated wt σ³⁸ promoter comprises the nucleotide sequence GCGG (SEQ ID NO 3), wherein in the stationary σ³⁸ promoter the nucleotide sequence GCGG (SEQ ID NO 3) is replaced by the nucleotide sequence AAAT (SEQ ID NO 4).

According to an embodiment (15), a microorganism is suggested, wherein the microorganism is selected from Bacteria and Archaea and a genome of the microorganism encompasses the nucleotide construct according to any of embodiments (13) and (14).

According to an embodiment (16), the microorganism according to embodiment (15) is selected from Bacteria, wherein a strain of the Bacteria is selected from: a Bacillus, a Bacillus subtilis, a Brevibacterium, a Chromohalobacter, a Corynebacterium glutamicum, an Escherichia coli, a Halobacillus, a Halomonas, a Halomonas elongata, a Marinococcus, and a Methylomicrobium.

According to an embodiment (17), a biotechnical process for continuous production of a natural compound by a microorganism is suggested, wherein a culture of the microorganism is maintained in an exponential growth phase or in a stationary phase, wherein a genome of the microorganism encompasses the nucleotide construct according to claim 13 or 14, wherein the microorganism is adapted to excrete the natural compound into a culture medium, wherein the process comprises extracting the natural compound from the culture medium.

According to an embodiment (18), in the process according to embodiment (17) the microorganism is Halomonas elongata, and the natural compound is selected from: Nγ-acetyl-L-2,4-diaminobutyrate, ectoine, and hydroxyectoine.

Although various exemplary embodiments of the invention have been disclosed, it will be apparent to those skilled in the art that various changes and modifications can be made which will achieve some of the advantages of the invention without departing from the spirit and scope of the invention. It will be obvious to those reasonably skilled in the art that other components performing the same functions may be suitably substituted. It should be mentioned that features explained with reference to a specific figure may be combined with features of other figures, even in those cases in which this has not explicitly been mentioned. Such modifications to the inventive concept are intended to be covered by the appended claims.

### REFERENCES

Espah Borujeni A., Channarasappa A S., and Salis H M. 2014. Translation rate is controlled by coupled trade-offs between site accessibility, selective RNA unfolding and sliding at upstream standby sites. Nucleic Acids Research 42 (4), 2646-2659.
Grammann K., Volke A., and Kunte H J. 2002. New type of osmoregulated solute transporter identified in halophilic members of the bacteria domain: TRAP transporter TeaABC mediates uptake of ectoine and hydroxyectoine in Halomonas elongata DSM 2581^{T}. Journal of Bacteriology 184 (11), 3078-3085.
Kovach M E., Elzer P H., Hill D S., Robertson G T., Farris M A., Roop R M 2nd, and Peterson K M. (1995) Four new derivatives of the broad-host-range cloning vector pBBR1MCS, carrying different antibiotic-resistance cassettes. Gene 166 (1), 175-176.
Kunte H J. 2006. Osmoregulation in bacteria: compatible solute accumulation and osmosensing. Environmental Chemistry 3, 94-99.
Kunte H J., Lentzen G., and Galinski E A. 2014. Industrial production of the cell protectant ectoine: protection, mechanisms, processes, and products. Current Biotechnology 3 (1), 1-16.
Larsen, P. I., Sydnes, L. K., Landfald, B., and Strom, A. R. 1987. Osmoregulation in Escherichia coli by accumulation of organic osmolytes: betaines, glutamic acid, and trehalose. Archive of Microbiology 147, 1-7.
Lee S J., and Gralla J D. (2001) Sigma38 (rpoS) RNA polymerase promoter engagement via −10 region nucleotides. The Journal of Biological Chemistry 276 (32), 30064-30071.
Lee S J., and Gralla J D. (2004) Osmo-regulation of bacterial transcription via poised RNA polymerase. Molecular Cell 14, 153-162.
Miller J H. 1972. Experiments in Molecular Genetics, Cold Spring Harbor Laboratory Press, Cold Spring Harbor, N Y.
Salis H M., Mirsky E A., and Voigt C A. 2009. Automated design of synthetic ribosome binding sites to control protein expression. Nature Biotechnology 27, 946-950.
Sambrook J., and Russell, R W. 2001. Molecular cloning: A laboratory manual, 3rd ed. Cold spring harbor laboratory press, cold spring harbor, N.Y.
Schwibbert K., Marin-Sanguino A., Bagyan I., Heidrich G., Lentzen G., Seitz H., Rampp M., Schuster S C., Klenk H P., Pfeiffer F., Oesterhelt D., and Kunte H J. 2011. A blueprint of ectoine metabolism from the genome of the industrial producer Halomonas elongata DSM 2581^{T}. Environmental Microbiology 13 (8), 1973-1994.

1. A method for increased and permanent production of a natural compound by a microorganism during an exponential growth and during a stationary phase of a culture of the microorganism, the method comprising:
enhancing a translation of a target mRNA, wherein the target mRNA comprises a transcript of a target gene, wherein the target gene is encoding the natural compound or a key enzyme involved in a biosynthesis of the natural compound in the microorganism, by replacing a wild type (wt) ribosome binding site (RBS) upstream of the target gene with a synthetic RBS, the synthetic RBS possessing a higher affinity towards a 16S rRNA of a ribosome of the microorganism than the wt RBS; and
enhancing a transcription of the target gene by converting an osmotically regulated σ³⁸ promoter upstream of the target gene into a stationary σ³⁸ promoter, wherein the osmotically regulated σ³⁸ promoter comprises a −35 G-element and a −10 element which are separated from each other by a spacer sequence, by deleting the −35 G element.

2. The method according to claim 1, wherein the −35 G element is deleted by replacing it with nucleotides others than guanine (G).

3. The method according to claim 1 or 2, further comprising:
cultivating the microorganism, a genome of the cultivated microorganism encompassing in a downstream direction the stationary σ³⁸ promoter, a σ⁷⁰ promoter, the synthetic RBS, and the target gene.

4. The method according to any of claims 1-3, wherein the natural compound is selected from a protein, an amino acid, an derivative of an amino acid, a sugar, and a sugar-polyol.

5. The method according to any of claims 1-4, wherein the microorganism is a halophilic microorganism and the natural compound is selected from a compatible solute, the compatible solute being selected from a sugar, a sugar-polyol, an amino acid and an amino acid derivative.

6. The method according to claim 4, wherein the natural compound is selected from Nγ-acetyl-L-2,4-diaminobutyrate, ectoine, and hydroxyectoine.

7. The method according to claim 6, wherein the microorganism is Halomonas elongata and the key enzyme is L-2,4-diaminobutyric acid Nγ-acetyltransferase.

8. The method according to claim 7, wherein the synthetic RBS comprises the nucleotide sequence CTAAGGAGAC (SEQ ID NO 1).

9. The method according to claim 8, wherein the synthetic RBS comprises the nucleotide sequence AGACTTATTCTAATCTAAGGAGACTACCC (SEQ ID NO 2).

10. The method according to claim 9, wherein the −35 G-element of the osmotically regulated σ³⁸ promoter comprises GCGG (SEQ ID NO 3), wherein converting the stationary regulated σ³⁸ promoter into the stationary σ³⁸ promoter comprises replacing GCGG (SEQ ID NO 3) with AAAT (SEQ ID NO 4).

11. The method according to claim 10, wherein the nucleotide sequence of the stationary σ³⁸ promoter comprises the nucleotide sequence CTACACT (SEQ ID NO 5).

12. The method according to claim 11, wherein the nucleotide sequence of the stationary σ³⁸ promoter comprises the nucleotide sequence
(SEQ ID NO 6)
TTTCTGCCAAATTCCATGAAATCGTCTACACT.

13. A nucleotide construct comprising:
a synthetic ribosome binding site (RBS),
a stationary σ³⁸ promoter,
a σ⁷⁰ promoter, and
a target gene;
the synthetic RBS possessing a higher affinity towards a 16S rRNA of a bacterial ribosome than a wild type (wt) RBS upstream of the target gene;
the stationary σ³⁸ promoter being generated from an osmotically regulated wt σ³⁸ promoter, the osmotically regulated wt σ³⁸ promoter comprising a −35 G-element, by deleting the −35 G-element and/or by replacing the −35 G-element by a nucleotide, the nucleotide being selected from nucleotides others than guanine;
wherein the target gene is encoding a key enzyme, the key enzyme catalyzing a reaction selected from: a phosphorylation/dephosphorylation, a carboxylation/decarboxylation, and an acetylation; the key enzyme being involved in a biosynthesis of a natural compound, wherein the natural compound is selected from a protein, an amino acid, a derivative of an amino acid, a sugar, and a sugar-polyol.

14. The nucleotide construct according to claim 13, wherein the −35 G-element of the osmotically regulated wt σ³⁸ promoter comprises GCGG (SEQ ID NO 3), in the stationary σ³⁸ promoter GCGG (SEQ ID NO 3) being replaced by AAAT (SEQ ID NO 4).

15. A microorganism, the microorganism being selected from Bacteria and Archaea and a genome of the microorganism encompassing the nucleotide construct according to claim 13 or 14.

16. The microorganism according to claim 15, wherein the microorganism is selected from Bacteria, a strain of the Bacteria being selected from: a Bacillus, a Bacillus subtilis, a Brevibacterium, a Chromohalobacter, a Corynebacterium glutamicum, an Escherichia coli, a Halobacillus, a Halomonas, a Halomonas elongata, a Marinococcus, and a Methylomicrobium.

17. A biotechnical process for continuous production of a natural compound by a microorganism, wherein a culture of the microorganism is maintained in an exponential growth phase or in a stationary phase, wherein a genome of the microorganism encompasses the nucleotide construct according to claim 13 or 14, wherein the microorganism is adapted to excrete the natural compound into a culture medium, the process comprising extracting the natural compound from the culture medium.

18. The process according to claim 17, wherein the microorganism is Halomonas elongata, and the natural compound is selected from Nγ-acetyl-L-2,4-diaminobutyrate, ectoine, and hydroxyectoine.
